(19) United States
(12) Patent Application Publication
(10) Pub. No.: US 2014/0120174 A1
(43) Pub. Date: May 1, 2014

(54) METHODS OF PROGNOSIS AND DIAGNOSIS OF SEPSIS

(71) Applicants: LASCCO, SA, Geneva (CH); Abbott Laboratories, Abbott Park, IL (US)

(72) Inventors: Stuart Blincko, Hessen (DE); Ingo Curdt, Hochheim (DE); Mario Berger, Wiesbaden (DE); Martin John Llewelyn, Brighton (GB)

(21) Appl. No.: 14/066,931

(22) Filed: Oct. 30, 2013

Related U.S. Application Data
(60) Provisional application No. 61/720,225, filed on Oct. 30, 2012.

(51) Int. Cl. G01N 33/68

(52) U.S. Cl. CPC G01N 33/6893
USPC 424/537; 435/7.92; 436/501; 514/1.4; 435/7.94

(57) ABSTRACT

Provided are methods of diagnosing sepsis in a patient by detecting the presence and/or amount of at least two biomarkers of sepsis or severe sepsis/septic shock in a sample from the patient. The methods and biomarkers may be used to develop an accurate prognosis for a patient having sepsis or severe sepsis/septic shock or suspected of having sepsis or severe sepsis/septic shock, or to accurately diagnose a patient having, or suspected of having sepsis or severe sepsis/septic shock. The methods and biomarkers may be used to identify and/or classify a patient as a candidate for a sepsis therapy.

The subject application claims priority to U.S. Provisional Application No. 61/720,225, filed on Oct. 30, 2012, hereby incorporated in its entirety by reference.

### FIELD OF THE INVENTION

The present invention relates to methods and immunoassay platforms for determining a prognosis, diagnosis, or risk identification of sepsis or severe sepsis/septic shock in a patient by detecting biomarkers in the patient as well as determining amounts thereof. The biomarkers may be used to identify a patient with sepsis or severe sepsis/septic shock, identify a patient as a candidate for sepsis therapy, to classify a patient's risk of developing severe sepsis or septic shock, or to classify a patient's sepsis stage or risk of progression of sepsis, as well as to determine a diagnosis, prognosis, or a treatment regimen.

### BACKGROUND

Sepsis is a common reason for admission to intensive care and high dependency units and the incidence of sepsis continues to rise. The symptoms and signs of sepsis are highly variable, which makes clinical recognition and assessment of severity difficult. At the point of admission to high dependency care, doctors are confronted with conflicting pressures. On one hand, there is a drive to minimize unnecessary antibiotic prescribing; and on the other, there is compelling evidence that timely and specific administration of treatment saves lives. Biomarkers may be used to inform decision makers regarding whether there is the presence or absence of infection, and its' severity in a patient who has a systemic inflammatory response syndrome (SIRS).

A recent review of the sepsis biomarker literature identified 178 biomarkers evaluated in 3370 studies. The issue or major challenge in evaluating new sepsis biomarkers is the lack of any gold-standard test. Consequently, early evaluation of biomarkers tends to involve very tightly clinically defined patient groups, which do not reflect the heterogeneity that clinicians encounter. It is unlikely that one markers would be sufficient to adequately diagnose, predict or stratify the severity of sepsis. This is due to the diversity of causes (eg bacterial, viral, fungal) and the multifactoral response of the host.

Some exemplary markers include C-reactive protein (CRP) that has been in use for over 20 years, but has poor specificity unless high cut-off levels are used. Procalcitonin (PCT) appears to be more specific for infection and, unlike CRP, levels appear to reflect with severity and prognosis. However, a recent meta-analysis challenged this and subsequent controlled trials of PCT-guided treatment of sepsis have produced conflicting results. Sepsis has also been associated with increase percentages of Treg cells and elevated plasma levels of soluble CD55 (sCD25). In addition, measurements of pancreatic stone protein (PSP) within 24 hours of ICU admission may predict in-hospital mortality in patients with septic shock. However, none of these studies are able to distinguish patients suffering from systemic inflammatory response syndrome without infection or patients suffering from an infection leading to sepsis, severe sepsis or septic shock and also showing signs of SIRS. Serum biomarker panels may provide a means for screening these targeted high risk individuals, which could greatly increase the survival rates, but particular combinations of these markers have not been identified. Accordingly, there remains a great need for more accurate and precise markers for sepsis, severe sepsis or septic shock.

### SUMMARY OF THE INVENTION

The present invention is directed to a method for identifying and treating a subject with sepsis or severe sepsis, the method comprising obtaining a biological sample comprising blood from the subject; determining the level of soluble CD25 (sCD25) and pancreatic stone protein (PSP) in the biological sample from the subject; comparing the levels of sCD25 and PSP in the biological sample to reference levels of sCD25 and PSP; identifying the subject as having sepsis or severe sepsis if the levels of sCD25 and PSP in the biological sample are greater than the reference levels of sCD25 and PSP; and administering a sepsis treatment regimen to the subject identified as having sepsis or severe sepsis. The reference levels of sCD25 and PSP are the levels of sCD25 and PSP in a control sample, or sCD25 and PSP cutoff values. The reference levels of sCD25 and PSP are the levels of sCD25 and PSP in a control sample, wherein the control sample is selected from the group consisting of a biological sample of a control subject and sCD25 and PSP concentration standards. The sCD25 and PSP concentration standards are selected from sCD25 and PSP concentration values of a control sample, and the median or the mean sCD25 and PSP concentrations of a plurality of samples from a group of control subjects. The determined levels of sCD25 and PSP of the subject may be at least double the reference levels of sCD25 and PSP. The reference levels of sCD25 and PSP may be the sCD25 and PSP cutoff values determined by a receiver operating curve (ROC) analysis from biological samples of a patient group. The reference levels of sCD25 and PSP may be the sCD25 and PSP cutoff values determined by a quartile analysis of biological samples of a patient group. The reference levels of sCD25 and PSP may be the sCD25 and PSP cutoff values, and may be about 2 ng/mL for sCD25 and about 25 ng/mL in serum for PSP. The subject may be a human. The reference levels of sCD25 and PSP may be from a biological sample of a human control subject. The biological sample of a subject is selected from a tissue sample, bodily fluid, whole blood, plasma, serum, urine, bronchoalveolar lavage fluid, cerebrospinal fluid and a cell culture suspension or fraction thereof. The levels of sCD25 and PSP comprise an immunological method with molecules binding to sCD25 and PSP. The molecules binding to sCD25 and PSP comprise at least one antibody capable of specifically binding sCD25 or PSP. The sepsis treatment regimen comprises administering at least one of an antibiotic, a vasopressor, a steroid, insulin, painkillers, sedatives, oxygen, and intravenous fluid to the subject. The method of invention may further comprise determining the level of at least one additional biomarker of sepsis in the biological sample, and comparing the level of the at least one additional biomarker of sepsis to a reference concentration value for the at least one biomarker of sepsis. The additional biomarker of sepsis is procalcitonin (PCT).

The present invention is also directed to a method of providing a diagnosis of a subject having sepsis, the method comprising the steps of obtaining a biological sample comprising blood from the subject; determining the level of soluble CD25 (sCD25) and pancreatic stone protein (PSP) in the biological sample from the subject; comparing the levels of sCD25 and PSP in the biological sample to reference levels of sCD25 and PSP; and providing a diagnosis of a subject having sepsis or severe sepsis if the levels of sCD25 and PSP in the biological sample are greater than the reference levels of sCD25 and PSP. The method of invention may further comprise administering a sepsis treatment regimen.

The present invention is also directed to a method of determining the severity of sepsis in a subject; the method comprising obtaining a biological sample comprising blood from the subject; determining the level of soluble CD25 (sCD25) and pancreatic stone protein (PSP) in the biological sample from the subject; comparing the levels of sCD25 and PSP in the biological sample to reference levels of sCD25 and PSP; and correlating the levels of sCD25 and PSP in the biological sample with severity of sepsis in the subject wherein if the levels of sCD25 and PSP in the biological sample are higher than the reference level of sCD25 and PSP in the biological sample, the subject is determined to have increased severity of sepsis. The method of invention may further comprise administering a sepsis treatment regimen.

The present invention is also directed to a method of monitoring the progression of sepsis in a subject, the method comprising obtaining a biological sample comprising blood from the subject; determining the level of soluble CD25 (sCD25) and pancreatic stone protein (PSP) in the biological sample from the subject; and correlating the levels of sCD25 and PSP with progression of sepsis in the subject wherein if the levels of sCD25 and PSP are higher as compared to the levels of sCD25 and PSP in an earlier biological sample from the subject, the subject is identified as having progression of sepsis. The method of invention may further comprise administering a sepsis treatment regimen.

The present invention is also directed to a method for the diagnosis, prognosis and/or risk stratification of sepsis or severe sepsis in a subject having or suspected of sepsis or severe sepsis, the method comprising the step of detecting increased levels of soluble CD25 (sCD25) and pancreatic stone protein (PSP) in the subject relative to a control subject not having sepsis or severe sepsis. The method of invention may further comprise administering a sepsis treatment regimen.

The present invention is also directed to a kit for performing the methods as described above. The kit may comprise at least one reagent capable of specifically binding sCD25 or PSP to quantify the levels of sCD25 or PSP in the biological sample of a subject; and a reference standard indicating reference levels of sCD25 and PSP. The at least one reagent may comprise at least one antibody capable of specifically binding sCD25 or PSP. The kit of invention may further comprise at least one additional reagent capable of binding at least one additional biomarker of sepsis in the biological sample to quantify the concentration of the at least one additional biomarker in the biological sample, and a reference standard indicating a reference concentration of the at least one additional biomarker of sepsis in the biological sample.

The present invention is directed to a method for identifying and treating a subject with sepsis or severe sepsis, the method comprising obtaining a biological sample comprising blood from the subject; determining the level of soluble CD25 (sCD25) and procalcitonin (PCT) in the biological sample from the subject; comparing the levels of sCD25 and PCT in the biological sample to reference levels of sCD25 and PCT; identifying the subject as having sepsis or severe sepsis if the levels of sCD25 and PCT in the biological sample are greater than the reference levels of sCD25 and PCT; and administering a sepsis treatment regimen to the subject identified as having sepsis or severe sepsis. The reference levels of sCD25 and PCT are the levels of sCD25 and PCT in a control sample, or sCD25 and PCT cutoff values. The reference levels of sCD25 and PCT are the levels of sCD25 and PCT in a control sample, wherein the control sample is selected from the group consisting of a biological sample of a control subject and sCD25 and PCT concentration standards. The sCD25 and PCT concentration standards are selected from sCD25 and PCT concentration values of a control sample, and the median or the mean sCD25 and PCT concentrations of a plurality of samples from a group of control subjects. The determined levels of sCD25 and PCT of the subject may be at least double the reference levels of sCD25 and PCT. The reference levels of sCD25 and PCT may be the sCD25 and PCT cutoff values determined by a receiver operating curve (ROC) analysis from biological samples of a patient group. The reference levels of sCD25 and PCT may be the sCD25 and PCT cutoff values determined by a quartile analysis of biological samples of a patient group. The reference levels of sCD25 and PCT may be the sCD25 and PCT cutoff values, and may be about 2 ng/mL for sCD25 and about 1 ng/mL in serum for PCT. The subject may be a human. The reference levels of sCD25 and PCT may be from a biological sample of a human control subject. The biological sample of a subject is selected from a tissue sample, bodily fluid, whole blood, plasma, serum, urine, bronchoalveolar lavage fluid, cerebrospinal fluid and a cell culture suspension or fraction thereof. The levels of sCD25 and PCT comprise an immunological method with molecules binding to sCD25 and PCT. The molecules binding to sCD25 and PCT comprise at least one antibody capable of specifically binding sCD25 or PCT. The sepsis treatment regimen comprises administering at least one of an antibiotic, a vasopressor, a steroid, insulin, painkillers, sedatives, oxygen, and intravenous fluid to the subject. The method of invention may further comprise determining the level of at least one additional biomarker of sepsis in the biological sample, and comparing the level of the at least one additional biomarker of sepsis to a reference concentration value for the at least one biomarker of sepsis. The additional biomarker of sepsis is pancreatic stone protein (PSP).

The present invention is also directed to a method of providing a diagnosis of a subject having sepsis, the method comprising the steps of obtaining a biological sample comprising blood from the subject; determining the level of soluble CD25 (sCD25) and procalcitonin (PCT) in the biological sample from the subject; comparing the levels of sCD25 and PCT in the biological sample to reference levels of sCD25 and PCT; and providing a diagnosis of a subject having sepsis or severe sepsis if the levels of sCD25 and PCT in the biological sample are greater than the reference levels of sCD25 and PCT. The method of invention may further comprise administering a sepsis treatment regimen.

The present invention is also directed to a method of determining the severity of sepsis in a subject; the method comprising obtaining a biological sample comprising blood from the subject; determining the level of soluble CD25 (sCD25) and procalcitonin (PCT) in the biological sample from the subject; comparing the levels of sCD25 and PCT in the biological sample to reference levels of sCD25 and PCT; and correlating the levels of sCD25 and PCT in the biological sample with severity of sepsis in the subject wherein if the levels of sCD25 and PCT in the biological sample are higher than the reference level of sCD25 and PCT in the biological sample, the subject is determined to have increased severity of sepsis. The method of invention may further comprise administering a sepsis treatment regimen.

The present invention is also directed to a method of monitoring the progression of sepsis in a subject, the method comprising obtaining a biological sample comprising blood from the subject; determining the level of soluble CD25 (sCD25) and procalcitonin (PCT) in the biological sample from the subject; and correlating the levels of sCD25 and PCT with progression of sepsis in the subject wherein if the levels of sCD25 and PCT are higher as compared to the levels of sCD25 and PCT in an earlier biological sample from the subject, the subject is identified as having progression of sepsis. The method of invention may further comprise administering a sepsis treatment regimen.

The present invention is also directed to a method for the diagnosis, prognosis and/or risk stratification of sepsis or severe sepsis in a subject having or suspected of sepsis or severe sepsis, the method comprising the step of detecting increased levels of soluble CD25 (sCD25) and procalcitonin (PCT) in the subject relative to a control subject not having sepsis or severe sepsis. The method of invention may further comprise administering a sepsis treatment regimen.

The present invention is also directed to a kit for performing the methods as described above. The kit may comprise at least one reagent capable of specifically binding sCD25 or PCT to quantify the levels of sCD25 or PCT in the biological sample of a subject; and a reference standard indicating reference levels of sCD25 and PCT. The at least one reagent may comprise at least one antibody capable of specifically binding sCD25 or PCT. The kit of invention may further comprise at least one additional reagent capable of binding at least one additional biomarker of sepsis in the biological sample to quantify the concentration of the at least one additional biomarker in the biological sample, and a reference standard indicating a reference concentration of the at least one additional biomarker of sepsis in the biological sample.

### BRIEF DESCRIPTION OF THE DRAWINGS

FIGS. 1A-1C show blood levels of biomarkers in patients with non-infectious SIRS or Sepsis at the time of admission to the intensive care or high dependency units. Boxes show Medians and IQRs, whiskers show 5th and 95th percentiles. FIG. 1A shows procalcitonin, FIG. 1B shows pancreatic stone protein, and FIG. 1C shows soluble CD25.

FIG. 2 shows the relationship between biomarker levels and severity of illness at the time of admission to ICU. Boxes show Medians and IQRs, whiskers show 5th and 95th percentiles.

### DETAILED DESCRIPTION

The present invention is directed to analyzing the levels of soluble CD25 (sCD25) and pancreatic stone protein (PSP) to identify, diagnose and treat sepsis or severe sepsis/septic shock in patients in need thereof. This method differs over previous sepsis diagnostic methods by using the unique combination of PSP and sCD25 markers to distinguish patients suffering from systemic inflammatory response syndrome (SNS) without infection and those with sepsis infection. The method may also distinguish patients suffering from sepsis infection and those with severe sepsis or septic shock (also referred to herein as “severe sepsis/septic shock”). In addition, the following combination of markers can be measured to decipher subjects with systemic inflammatory response syndrome (SIRS) with or without infection, or subjects with sepsis from subjects with severe sepsis or septic shock, by quantifying the levels of the following combination of markers selected from the group consisting of sCD25 and procalcitonin (PCT) and PSP and PCT.

Section headings as used in this section and the entire disclosure herein are merely for organizational purposes and are not intended to be limiting.

### A. DEFINITIONS

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The use of “or” means “and/or” unless stated otherwise. Furthermore, the use of the terms “including” and “having,” as well as other forms of those terms, such as “includes,” “included”, “has,” and “have” are not limiting.

The “confidence interval” or “CI” as used herein refers to an interval estimate of a population parameter used to indicate the reliability of an estimate. The confidence interval refers to the region containing the limits or band of a parameter with an associated confidence level that the bounds are large enough to contain the true parameter value. The bands may be single-sided to describe an upper or lower limit or double sided to describe both upper and lower limits. The region gives a range of values, bounded below by a lower confidence limit and from above by an upper confidence limit, such that one can be confident (at a pre-specified level such as 95% or 99%) that the true population parameter value is included within the confidence interval. Confidence intervals may be formed for any of the parameters used to describe the characteristic of interest. Confidence intervals may be used to estimate the population parameters from the sample statistics and allow a probabilistic quantification of the strength of the best estimate. A preferred confidence interval may be at least 90%, at least 91%, at least 92%, at least 93%, at least 94%, at least 95%, at least 96%, at least 97%, at least 98%, or at least 99%.

“Correlation coefficient” as used herein means a significant correlation may be determined by any suitable statistic method. For example, the correlation coefficient may be Spearman's rank correlation coefficient (also known as “Spearman's rho” and “Spearman's rho correlation coefficient”), which is a non-parametric measure of statistical dependence between two variables. Spearman's rho assesses how well the relationship between two variables can be described using a monotonic function. If there are no repeated data values, a perfect Spearman correlation of +1 or −1 occurs when each of the variables is a perfect monotone function of the other. A highly significant correlation is indicated when Spearman's rho is at least 0.50, preferably at least 0.60, more preferably at least 0.70, even more preferably at least 0.80, yet more preferably at least 0.85, even more preferably at least 0.90. Spearman's rho may be between 0.55 and 0.60. Most preferably, for two markers, Spearman's rho is at least approximately 0.50, at least approximately 0.55, at least approximately 0.60, at least approximately 0.65, at least approximately 0.70, at least approximately 0.75, at least approximately 0.80, at least approximately 0.85, at least approximately 0.90, at least approximately 0.91, at least approximately 0.92, at least approximately 0.93, at least approximately 0.94, at least approximately 0.95, at least approximately 0.96, at least approximately 0.97, at least approximately 0.98, or at least approximately 0.99. For example, sCD25 and PSP may have a Spearman's rho of at least approximately 0.584 and sCD25 and PCT may have a Spearman's rho of at least approximately 0.546.

“Infection” as used herein means microbial phenomenon characterized by an inflammatory response to the presence of microorganisms or the invasion of normally sterile host tissue by those organisms (see “Definitions for Sepsis and Organ Failure and Guidelines for the Use of Innovative Therapies in Sepsis” from the ACCP/SCCM consensus conference committee (Bone et al., Chest 1992, 101; 1644-1655), which is incorporated herein by reference in its entirety).

“Interquartile range” or “IQR” as used herein means a measure of statistical dispersion, being equal to the difference between the upper and lower quartiles, IQR=Q3−Q1.

“Non-infectious SIRS” or “SIRS without infection” as used herein refers to SIRS associated with an established underlying non-infectious diagnosis and no reason to suspect any on-going infection. SIRS is manifested by two or more of the following conditions: (1) temperature greater than 38° C. or less than 36° C.; (2) heart rate greater than 90 beats per minute; (3) respiratory rate greater than 20 breaths per minute or PaCO₂ less than 32 mm HG; and (4) white blood cell count greater than 12,000/cu mm, less than 4,000/cu mm, or less than 10% immature (band) forms (see “Definitions for Sepsis and Organ Failure and Guidelines for the Use of Innovative Therapies in Sepsis” from the ACCP/SCCM consensus conference committee (Bone et al., Chest 1992, 101; 1644-1655)).

“Predetermined cutoff” and “predetermined level” as used herein refers to an assay cutoff value that is used to assess diagnostic, prognostic, or therapeutic efficacy results by comparing the assay results against the predetermined cutoff/level, where the predetermined cutoff/level already has been linked or associated with various clinical parameters (e.g., presence of disease, stage of disease, severity of disease, progression, non-progression, or improvement of disease, etc.). The disclosure provides exemplary predetermined levels. However, it is well-known that cutoff values may vary depending on the nature of the immunoassay (e.g., antibodies employed, reaction conditions, sample purity, etc.). It further is well within the ordinary skill of one in the art to adapt the disclosure herein for other immunoassays to obtain immunoassay-specific cutoff values for those other immunoassays based on the description provided by this disclosure. Whereas the precise value of the predetermined cutoff/level may vary between assays, the correlations as described herein should be generally applicable.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “test sample,” “specimen,” “sample from a subject,” and “patient sample” as used herein may be used interchangeable and may be a sample of blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, bronchoalveolar lavage (BAL) fluid, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

“SOFA (sequential or Sepsis-related Organ Failure Assessment)” score as used herein means that it is a six-organ dysfunction/failure score measuring multiple organ failure daily. Each organ is graded from 0 (normal) to 4 (the most abnormal), providing a daily score of 0 to 24 points.

Sequential assessment of organ dysfunction during the first few days of ICU admission is a good indicator of prognosis. Both the mean and highest SOFA scores are particularly useful predictors of outcome. Independent of the initial score, an increase in SOFA score during the first 48 hours in the ICU predicts a mortality rate of at least 50%.

“Sepsis” as used herein is a condition characterized by a whole-body inflammatory state that is triggered by either a proven (on the basis of sampling or radiology) or probable (considering the patient's clinical presentation, white cell count, CRP, radiology) infection. The infection may be cause by bacteria, virus or fungi. Triggers of sepsis include pneumonia, such as ventilator-associated pneumonia, abdominal infection, kidney infection, and bloodstream infection. The body may develop this inflammatory response by the immune system to microbes in the blood, urine, lungs, skin, or other tissues. A lay term for sepsis is blood poisoning, also used to describe septicaemia. Septicaemia is a related medical term referring to the presence of pathogenic organisms in the bloodstream, leading to sepsis.

Symptoms related to the provoking infection, sepsis is characterized by presence of acute inflammation present throughout the entire body, and is, therefore, frequently associated with fever and elevated white blood cell count (leukocytosis) or low white blood cell count (leukopenia) and lower-than-average temperature, and vomiting. The modern concept of sepsis is that the host's immune response to the infection causes most of the symptoms of sepsis, resulting in hemodynamic consequences and damage to organs. This immunological response causes widespread activation of acute-phase proteins, affecting the complement system and the coagulation pathways, which then cause damage to the vasculature as well as to the organs. Various neuroendocrine counter-regulatory systems are then activated as well, often compounding the problem. Even with immediate and aggressive treatment, this may progress to multiple organ dysfunction syndrome and eventually death. Sepsis is manifested by two or more of the following conditions as a result of infection: (1) temperature greater than 38° C. or less than 36° C.; (2) heart rate greater than 90 beats per minute; (3) respiratory rate greater than 20 breaths per minute or PaCO₂ less than 32 mm HG; and (4) white blood cell count greater than 12,000/cu mm, less than 4,000/cu mm, or less than 10% immature (band) forms (see “Definitions for Sepsis and Organ Failure and Guidelines for the Use of Innovative Therapies in Sepsis” from the ACCP/SCCM consensus conference committee (Bone et al., Chest 1992, 101; 1644-1655)).

“Septic shock” as used herein is defined as sepsis with refractory hypotension or hypoperfusion abnormalities in spite of adequate fluid resuscitation along with the presence of perfusion abnormalities, which may include but are not limited to, lactic acidosis, oliguria, or an acute alteration in mental status (see “Definitions for Sepsis and Organ Failure and Guidelines for the Use of Innovative Therapies in Sepsis” from the ACCP/SCCM consensus conference committee (Bone et al., Chest 1992, 101; 1644-1655)). Patients with septic shock may have a cardiovascular SOFA score of 4.

“Severe sepsis” as used herein is the systemic inflammatory response to infection and the presence of organ dysfunction, hypoperfusion or hypotension. Hypoperfusion and perfusion abnormalities may include, but are not limited to lactic acidosis, oliguria, or an acute alteration in mental status (see “Definitions for Sepsis and Organ Failure and Guidelines for the Use of Innovative Therapies in Sepsis” from the ACCP/SCCM consensus conference committee (Bone et al., Chest 1992, 101; 1644-1655)). Severe sepsis may be defined as sepsis plus organ failure. Patients with sever sepsis may have a SOFA score of 2 or more for any organ system. Severe sepsis is usually treated in the intensive care unit with intravenous fluids and antibiotics. If fluid replacement isn't sufficient to maintain blood pressure, specific vasopressor medications can be used. Mechanical ventilation and dialysis may be needed to support the function of the lungs and kidneys, respectively. To guide therapy, a central venous catheter and an arterial catheter may be placed; measurement of other hemodynamic variables (such as cardiac output, mixed venous oxygen saturation, or stroke volume variation) may also be used. Sepsis patients require preventive measures for deep vein thrombosis, stress ulcers and pressure ulcers, unless other conditions prevent this. Some patients might benefit from tight control of blood sugar levels with insulin (targeting stress hyperglycemia). The use of corticosteroids (low dose or otherwise) is controversial. Activated drotrecogin alfa (recombinant protein C) has not been found to be helpful, and has recently been withdrawn from sale.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### B. METHOD OF USING A COMBINATION OF MARKERS FOR SEPSIS THERAPY

a. SIRS with Infection

The present invention is directed to a method for distinguishing a subject suffering from systemic inflammatory response syndrome (SIRS) with or without infection by quantifying the levels of the following combination of markers selected from the group consisting of soluble CD25 (sCD25) and pancreatic stone protein (PSP) and sCD25 and procalcitonin (PCT). Those subjects suffering from SIRS with an infection are developing or are already suffering from sepsis.

The method includes the steps of (1) obtaining a biological sample from a subject, (2) determining the level of sCD25 and PSP or sCD25 and PCT in the biological sample, (3) comparing the levels of sCD25 and PSP or sCD25 and PCT in the biological sample to a reference level of the respective combination of markers (i.e., sCD25 and PSP in the biological sample is compared to the reference level of sCD25 and PSP etc.), (4) identifying the subject as having sepsis or severe sepsis/septic shock if the levels of sCD25 and PSP or sCD25 and PCT in the biological sample are greater than the reference level of the corresponding marker; and (5) administering a sepsis treatment regiment to the subject identified as having sepsis or severe sepsis.

b. Severe Sepsis/Septic Shock

The present invention is further directed to a method for determining the severity of sepsis to a point of distinguishing between severe sepsis/sepsis shock and SIRS with infection by quantifying the levels of the following combination of markers selected from the group consisting of soluble CD25 (sCD25) and pancreatic stone protein (PSP) and sCD25 and procalcitonin (PCT). Those subjects suffering from SIRS with an infection are developing or are already suffering from sepsis.

The method includes the steps of (1) obtaining a biological sample from a subject, (2) determining the level of sCD25 and PSP or sCD25 and PCT in the biological sample, (3) comparing the levels of sCD25 and PSP or sCD25 and PCT in the biological sample to a reference level of the respective combination of markers (i.e., sCD25 and PSP in the biological sample is compared to the reference level of sCD25 and PSP etc.), (4) identifying the subject as having severe sepsis or septic shock if the levels of sCD25 and PSP or sCD25 and PCT in the biological sample are greater than the reference level of the corresponding marker; and (5) administering a sepsis treatment regiment to the subject identified as having severe sepsis or septic shock.

1. Method of Using Combination of Scd25 and Psp

a. SIRS with Infection

The method may be used to distinguish a subject suffering from systemic inflammatory response syndrome (SIRS) with or without infection by quantifying the levels of soluble CD25 (sCD25) and pancreatic stone protein (PSP) in a biological sample. The method includes the steps of (1) obtaining a biological sample from a subject, (2) determining the level of sCD25 and PSP in the biological sample, (3) comparing the levels of sCD25 and PSP in the biological sample to a reference level of sCD25 and PSP, (4) identifying the subject as having sepsis or severe sepsis if the levels of sCD25 and PSP in the biological sample are greater than the reference level of sCD25 and PSP, and (5) administering a sepsis treatment regimen to the subject identified as having sepsis or severe sepsis.

The reference level in this method can be the level of sCD25 and PSP in a SIRS patient who does not have an infection. Levels higher than or equal to 1.0 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, 2.0 ng/mL, 2.1 ng/mL, 2.2 ng/mL, 2.3 ng/mL, 2.4 ng/mL, 2.5 ng/mL, or 2.6 ng/mL of sCD25 in serum in combination with levels higher than or equal to 20 ng/mL, 21 ng/mL, 22 ng/mL, 23 ng/mL, 24 ng/mL, 25 ng/mL, 26 ng/mL, 27 ng/mL, 28 ng/mL, 29 ng/mL, or 30 ng/mL of PSP in serum identify the subject as having sepsis and suffering from SIRS as well. sCD25 and PSP perform as least comparably with procalcitonin (PCT) alone in identifying patients with sepsis, but with the added advantage of deciphering those subjects with SIRS by an infection and those with SIRS without an infection. Accordingly, subjects with SIRS due to trauma, burns, pancreatitis, ischemia, and hemorrhage, complications of surgery, adrenal insufficiency, pulmonary embolism, complicated aortic aneury, complications of surgery, adrenal insufficiency, pulmonary embolism, complicated aortic aneurysm, cardiac tamponade, anaphylaxis and drug overdose will be distinguished over those subjects with SIRS with infection. Accordingly, a physician can treat the subject with SIRS without infection or with infection with the appropriate treatment regimen.

b. Severe Sepsis/Septic Shock

The method may be used to distinguish a subject suffering from severe sepsis or septic shock by quantifying the levels of soluble CD25 (sCD25) and pancreatic stone protein (PSP) in a biological sample. The method includes the steps of (1) obtaining a biological sample from a subject, (2) determining the level of sCD25 and PSP in the biological sample, (3) comparing the levels of sCD25 and PSP in the biological sample to a reference level of sCD25 and PSP, (4) identifying the subject as having severe sepsis or septic shock if the levels of sCD25 and PSP in the biological sample are greater than the reference level of sCD25 and PSP, and (5) administering a sepsis treatment regimen to the subject identified as having severe sepsis or septic shock.

The reference level in this method can be the level of sCD25 and PSP in a patient suffer from sepsis. Levels higher than or equal to the reference level in serum identify the subject as having severe sepsis/septic shock. sCD25 and PSP perform as least comparably with procalcitonin (PCT) alone in identifying patients with severe sepsis/septic shock, but with the added advantage of deciphering those subjects with severe sepsis/septic shock and those with SIRS by an infection. Accordingly, a physician can treat the subject with severe sepsis/septic shock or SIRS with infection with the appropriate treatment regimen.

c. Non-SIRS with No Infection

The reference level in this method can be the level of sCD25 and PSP in a non-SIRS subject who does not have an infection. The reference level in this method can be a level lower than 0.9 ng/mL, 0.8 ng/mL, 0.7 ng/mL, 0.6 ng/mL, 0.5 ng/mL, 0.4 ng/mL, 0.3 ng/mL, 0.2 ng/mL, 0.1 ng/mL of sCD25 in serum in combination with levels lower than 10 ng/mL, 11 ng/mL, 12 ng/mL, 13 ng/mL, 14 ng/mL, 15 ng/mL, or 16 ng/mL of PSP in serum to identify a subject as being free of SIRS or SIRS with infection. This subject would be considered a non-SIRS healthy subject.

2. Method of Using Combination of sCD25 and PCT

a. SIRS with Infection

The method may be used to distinguish a subject suffering from systemic inflammatory response syndrome (SIRS) with or without infection by quantifying the levels of soluble CD25 (sCD25) and procalcitonin (PCT) in a biological sample. The method includes the steps of (1) obtaining a biological sample from a subject, (2) determining the level of sCD25 and PCT in the biological sample, (3) comparing the levels of sCD25 and PCT in the biological sample to a reference level of soluble sCD25 and PCT, (4) identifying the subject as having sepsis or severe sepsis if the levels of soluble sCD25 and PCT in the biological sample are greater than the reference level of sCD25 and PCT, and (5) administering a sepsis treatment regiment to the subject identified as having sepsis or severe sepsis.

The reference level in this method can be the level of sCD25 and PCT in a SIRS patient who does not have an infection. Levels higher than or equal to 1.0 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, 2.0 ng/mL, 2.1 ng/mL, 2.2 ng/mL, 2.3 ng/mL, 2.4 ng/mL, 2.5 ng/mL, or 2.6 ng/mL of sCD25 in serum in combination with levels higher or equal to 0.5 ng/mL, 0.6 ng/mL, 0.7 ng/mL, 0.8 ng/mL, 0.9 ng/mL, 1.0 ng/mL, 1.1 ng/mL, 1.2 ng/mL, 1.3 ng/mL, 1.4 ng/mL or 1.5 ng/mL of PCT in serum identify the subject as having sepsis and suffering from SIRS as well. The measurement of sCD25 and PCT provide this distinct distinction between subjects suffering from non-infection based SIRS and infection based SIRS.

b. Severe Sepsis/Septic Shock

The method may be used to distinguish a subject suffering from severe sepsis or septic shock by quantifying the levels of soluble CD25 (sCD25) and procalcitonin (PCT) in a biological sample. The method includes the steps of (1) obtaining a biological sample from a subject, (2) determining the level of sCD25 and PCT in the biological sample, (3) comparing the levels of sCD25 and PCT in the biological sample to a reference level of sCD25 and PCT, (4) identifying the subject as having severe sepsis or septic shock if the levels of sCD25 and PCT in the biological sample are greater than the reference level of sCD25 and PCT, and (5) administering a sepsis treatment regimen to the subject identified as having severe sepsis or septic shock.

The reference level in this method can be the level of sCD25 and PCT in a patient suffer from sepsis. Levels higher than or equal to the reference level in serum identify the subject as having severe sepsis/septic shock. sCD25 and PCT perform as least comparably with PCT alone in identifying patients with severe sepsis/septic shock, but with the added advantage of deciphering those subjects with severe sepsis/septic shock and those with SIRS by an infection. Accordingly, a physician can treat the subject with severe sepsis/septic shock or SIRS with infection with the appropriate treatment regimen.

c. Non-SIRS with No Infection

The reference level in this method can be the level of sCD25 and PCT in a non-SIRS subject who does not have an infection. The reference level in this method can be a level lower than 0.9 ng/mL, 0.8 ng/mL, 0.7 ng/mL, 0.6 ng/mL, 0.5 ng/mL, 0.4 ng/mL, 0.3 ng/mL, 0.2 ng/mL, 0.1 ng/mL of sCD25 in serum in combination with levels lower than 0.1 ng/mL, 0.2 ng/mL, 0.3 ng/mL, or 0.4 ng/mL of PCT in serum to identify a subject as being free of SIRS or SIRS with infection. This subject would be considered a non-SIRS healthy subject.

3. Systemic Inflammatory Response Syndrome (SIRS)

The method described above identifies those subjects with SIRS and whether they are infected or not. If infected, those subjects are suffering from sepsis. “Systemic inflammatory response syndrome” (SIRS) refers to a systemic host response to infection with SIRS plus a documented infection (e.g., a subsequent laboratory confirmation of a clinically significant infection such as a positive culture for an organism.

SIRS is defined by the presence of two or more of the following findings: Body temperature<36° C. (96.8° F.) or >38° C. (100.4° F.) (hypothermia or fever); Heart rate>90 beats per minute; Respiratory rate>20 breaths per minute or, on blood gas, a PaCO2 less than 32 mm Hg (4.3 kPa) (tachypnea or hypocapnia due to hyperventilation); White blood cell count <4,000 cells/mm3 or >12,000 cells/mm3 (<4×109 or >12×109 cells/L), or greater than 10% band forms (immature white blood cells) (leukopenia, leukocytosis, or bandemia). SIRS may be broadly classified as infectious or noninfectious. As above, when SIRS is due to an infection, it is considered sepsis. Noninfectious causes of SIRS include trauma, burns, pancreatitis, ischemia, and hemorrhage, complications of surgery, adrenal insufficiency, pulmonary embolism, complicated aortic aneury, complications of surgery, adrenal insufficiency, pulmonary embolism, complicated aortic aneurysm, cardiac tamponade, anaphylaxis and drug overdose.

4. Controls

a. Controls for SIRS with or without Infection

The method for distinguishing a subject suffering from systemic inflammatory response syndrome (SIRS) with or without infection is accomplished by quantifying the levels of the following combination of markers selected from the group consisting of soluble CD25 (sCD25) and pancreatic stone protein (PSP) and sCD25 and procalcitonin (PCT).

b. Controls for Severe Sepsis/Septic Shock

The method for distinguishing a subject suffering from sepsis or severe sepsis/septic shock is accomplished by quantifying the levels of the following combination of markers selected from the group consisting of soluble CD25 (sCD25) and pancreatic stone protein (PSP) and sCD25 and procalcitonin (PCT).

c. Pancreatic Stone Protein (PSP)

PSP may be used in the methods described above in combination with sCD25. PSP, also known as regenerating protein and lithosatathine, is a lectin-binding protein, the blood levels of which increase in inflammation. Among trauma patients, levels of PSP rise when sepsis develops. PSP levels at the onset of ventilator associated pneumonia have been shown to correlate with survival.

d. Soluble CD25 (sCD25)

Soluble CD25 (sCD25) may be used in the methods described above in combination with PSP and PCT. sCD25 is the IL2 receptor alpha chain and expressed constitutively on regulatory, FOXP3+, T cells. It is also expressed on effector T cells following activation and may reflect the development of a compensatory regulatory response. sCD25 has been found to be higher in patients with sepsis than in patients with non-infectious SIRS.

e. Procalcitonin (PCT)

Procalcitonin may be used in the methods described above in combination with sCD25. Procalcitonin (PCT) is a peptide precursor of the hormone calcitonin, the latter being involved with calcium homeostasis. It is composed of 116 amino acids and is produced by parafollicular cells (C cells) of the thyroid and by the neuroendocrine cells of the lung and the intestine. The level of procalcitonin rises in a response to a proinflammatory stimulus, especially of bacterial origin. In this case, it is produced mainly by the cells of the lung and the intestine. It does not rise significantly with viral or non-infectious inflammations. In serum, procalcitonin has a half-life of 25 to 30 hours. Remarkably the high procalcitonin levels produced during infections are not followed by a parallel increase in calcitonin or serum calcium levels.

6. Reference Level

a. SIRS with Infection

The method uses reference levels to determine whether an individual is suffering from SIRS with infection, SIRS without infection, or is a healthy subject with no SIRS. The reference levels of the various combinations of the method described above determines whether a subject is suffering from SIRS without infection or SIRS with infection, or a healthy subject that is free of SIRS as well. The reference level of markers used in the particular combinations discussed above may be a predetermined cutoff value, or a level determined from a control subject expressing the markers discussed above wherein that control subject is know to be a healthy non-SIRS subject or a SIRS without infection subject. Cutoff values (or predetermined cutoff value) may be determined by a receiver operating curve (ROC) analysis from biological samples of a patient group. ROC analysis, as generally known in the biological arts, is a determination of the ability of a test to discriminate one condition from another, e.g. to determine the performance of each marker in identifying sepsis or severe sepsis. A description of ROC analysis as applied according to the present disclosure is provided in P. J. Heagerty et al., Time-dependent ROC curves for censored survival data and a diagnostic marker, Biometrics 56:337-44 (2000), the disclosure of which is hereby incorporated by reference in its entirety.

Alternatively, cutoff values can be determined by a quartile analysis of biological samples of a patient group. For example, a cutoff value can be determined by selecting a value that corresponds to any value in the 25th-75th percentile range, preferably a value that corresponds to the 25th percentile, the 50th percentile or the 75th percentile, and more preferably the 75th percentile. For example, the cutoff value for sCD25 may be at least 1.0 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, 2.0 ng/mL, 2.1 ng/mL, 2.2 ng/mL, 2.3 ng/mL, 2.4 ng/mL, 2.5 ng/mL, or 2.6 ng/mL of sCD25 in serum. The cutoff value for PSP may be at least about 20 ng/mL, 21 ng/mL, 22 ng/mL, 23 ng/mL, 24 ng/mL, 25 ng/mL, 26 ng/mL, 27 ng/mL, 28 ng/mL, 29 ng/mL, or 30 ng/mL. The cutoff value for PCT may be at least about 1.0 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, 2.0 ng/mL, 2.1 ng/mL, 2.2 ng/mL, 2.3 ng/mL, 2.4 ng/mL, 2.5 ng/mL, or 2.6 ng/mL. An exemplary reference value obtained from the median of a relevant patient group is about 2.0 ng/mL for sCD25 and 25.0 ng/mL for PSP.

Such statistical analyses can be performed using any method known in the art and can be implemented through any number of commercially available software packages (e.g., from Analyse-it Software Ltd., Leeds, UK; StataCorp LP, College Station, Tex.; SAS Institute Inc., Cary, N.C.).

The “area under curve” or “AUC” refers to area under a ROC curve. AUC under a ROC curve is a measure of accuracy. An area of 1 represents a perfect test, whereas an area of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

b. Severe Sepsis/Septic Shock

The method uses reference levels to determine whether an individual is suffering from sepsis or severe sepsis/septic shock. The reference levels of the various combinations of the method described above determines whether a subject is suffering from sepsis or severe sepsis/septic shock. The reference level of markers used in the particular combinations discussed above may be a predetermined cutoff value, or a level determined from a control subject expressing the markers discussed above wherein that control subject is known to be a sepsis subject. Cutoff values (or predetermined cutoff value) may be determined by a receiver operating curve (ROC) analysis from biological samples of a patient group. ROC analysis, as generally known in the biological arts, is a determination of the ability of a test to discriminate one condition from another, e.g. to determine the performance of each marker in identifying sepsis or severe sepsis. A description of ROC analysis as applied according to the present disclosure is provided in P. J. Heagerty et al., Time-dependent ROC curves for censored survival data and a diagnostic marker, Biometrics 56:337-44 (2000), the disclosure of which is hereby incorporated by reference in its entirety.

Alternatively, cutoff values can be determined by a quartile analysis of biological samples of a patient group. For example, a cutoff value can be determined by selecting a value that corresponds to any value in the 25th-75th percentile range, preferably a value that corresponds to the 25th percentile, the 50th percentile or the 75th percentile, and more preferably the 75th percentile.

Such statistical analyses can be performed using any method known in the art and can be implemented through any number of commercially available software packages (e.g., from Analyse-it Software Ltd., Leeds, UK; StataCorp LP, College Station, Tex.; SAS Institute Inc., Cary, N.C.).

The “area under curve” or “AUC” refers to area under a ROC curve. AUC under a ROC curve is a measure of accuracy. An area of 1 represents a perfect test, whereas an area of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

7. Combination Method

The method as described above may further comprise measuring a third or more biomarker in combination with the specific combinations discussed above to determine whether a subject is suffering from sepsis or severe sepsis/septic shock. The method may further comprise determining the level of at least one additional biomarker of sepsis in the biological sample and comparing the level of the at least one additional biomarker of sepsis or severe sepsis/septic shock to a reference concentration value for the at least one biomarker of sepsis or severe sepsis/septic shock. The additional biomarker may be procalcitonin (PCT), sCD25 and PSP. For example, the method may further comprise determining the level of PCT in the biological sample and comparing the level of PCT in the biological sample to a reference level of PCT, wherein levels of sCD25, PSP, and PCT in the biological sample greater than the reference levels of sCD25, PSP, and PCT identifies the subject as having sepsis or severe sepsis/septic shock.

### C. METHOD OF PROVIDING A DIAGNOSIS OF A SUBJECT HAVING SEPSIS OR SEVERE SEPSIS/SEPTIC SHOCK

The present invention is also directed to a method for providing a diagnosis of a subject having sepsis or severe sepsis/septic shock by quantifying the levels of the following combination of markers selected from the group consisting of soluble CD25 (sCD25) and pancreatic stone protein (PSP) and sCD25 and procalcitonin (PCT). Those subjects suffering from sepsis with an infection are developing or are already suffering from sepsis.

The method includes the steps of (1) obtaining a biological sample from a subject, (2) determining the level of sCD25 and PSP or sCD25 and PCT in the biological sample, (3) comparing the levels of sCD25 and PSP or sCD25 and PCT in the biological sample to a reference level of the respective combination of markers (i.e., sCD25 and PSP in the biological sample is compared to the reference level of sCD25 and PSP etc.), (4) providing a diagnosis of a subject having sepsis or severe sepsis/septic shock if the levels of sCD25 and PSP or sCD25 and PCT in the biological sample are greater than the reference level of the corresponding marker; and (5) administering a sepsis treatment regiment to the subject identified as having sepsis or severe sepsis/septic shock. The reference levels of sCD25, PSP, or PCT are the same as those levels of SIRS with infection for determining sepsis and levels of severe sepsis/septic shock, as disclosed above.

### D. METHOD OF DETERMINING THE SEVERITY OF SEPSIS IN A SUBJECT

The present invention is also directed to a method for determining the severity of sepsis in a subject by quantifying the levels of the following combination of markers selected from the group consisting of soluble CD25 (sCD25) and pancreatic stone protein (PSP) and sCD25 and procalcitonin (PCT). Those subjects suffering from sepsis with an infection are developing or are already suffering from sepsis.

The method includes the steps of (1) obtaining a biological sample from a subject, (2) determining the level of sCD25 and PSP or sCD25 and PCT in the biological sample from the subject, (3) comparing the levels of sCD25 and PSP or sCD25 and PCT in the biological sample to a reference level of the respective combination of markers (i.e., sCD25 and PSP in the biological sample is compared to the reference level of sCD25 and PSP etc.), (4) correlating the levels of sCD25 and PSP or sCD25 and PCT in the biological sample to the reference level of the corresponding marker; and if the levels of particular combination of markers is higher than the reference levels of the same combination of markers, the subject is determined to have increased severity of sepsis.

### E. METHOD FOR MONITORING THE PROGRESSION OF SEPSIS IN A SUBJECT

The present invention is also directed to a method for monitoring the progression of sepsis in a subject by quantifying the levels of the following combination of markers selected from the group consisting of soluble CD25 (sCD25) and pancreatic stone protein (PSP) or sCD25 and procalcitonin (PCT). Those subjects suffering from sepsis with an infection are developing or are already suffering from sepsis.

The method includes the steps of (1) obtaining a biological sample from a subject, (2) determining the level of sCD25 and PSP or sCD25 and PCT in the biological sample from the subject, (3) correlating the levels of sCD25 and PSP or sCD25 and PCT with the progression of sepsis in the subject wherein if the levels of these combination of markers is higher as compared to the levels of these combination of markers in an earlier biological sample from the subject, the subject is identified as having progression of sepsis.

### F. METHOD FOR DIAGNOSIS, PROGNOSIS, AND/OR RISK STRATIFICATION OF SEPSIS OR SEVERE SEPSIS/SEPTIC SHOCK

The present invention is also directed to a method for the diagnosis, prognosis, and/or risk stratification of sepsis or severe sepsis/septic shock in a subject having or suspected of sepsis or severe sepsis/septic shock by quantifying the levels of the following combination of markers selected from the group consisting of soluble CD25 (sCD25) and pancreatic stone protein (PSP) and sCD25 and procalcitonin (PCT). Those subjects suffering from sepsis with an infection are developing or are already suffering from sepsis.

The method includes the steps of (1) obtaining a biological sample from a subject, (2) determining the level of sCD25 and PSP or sCD25 and PCT in the biological sample from the subject, (3) comparing the levels of sCD25 and PSP or sCD25 and PCT in the biological sample to a levels of the respective combination of markers (i.e., sCD25 and PSP in the biological sample is compared to the reference level of sCD25 and PSP) with a subject not having sepsis or severe sepsis. The method may further comprise the step of a treatment regiment.

### G. TREATMENT OF SUBJECTS SUFFERING FROM SIRS AND SEPSIS OR SEVERE SEPSIS/SEPTIC SHOCK

The subject identified in the methods described above having levels of soluble CD25 (sCD25) and PSP or sCD25 and PCT over the values discussed above are identified as patients suffering from sepsis or severe sepsis/septic shock. The subjects are then treated for the sepsis or severe sepsis/septic shock. Treatment may include being administered oxygen, either by a tube that is placed near the nose or through a clear plastic mask. Depending on the results of the tests, the physician may order medications. These medications may include antibiotics given intravenously (given directly into the vein). Initially, the antibiotics may be those that kill many different bacteria because the exact kind of infection the patient has is not known. Once the blood culture results show the identity of the bacteria, the doctor may select a different antibiotic that kills the specific organism responsible for the infection. The doctor may also order IV salt solution saline and medications to increase the blood pressure it is too low. The patient may be admitted to the hospital at least until the blood culture results are known. If the patient is very ill and with low blood pressure, the doctor may admit the patient to the intensive care unit (ICU) and may consult specialist doctors to help in the management of the illness. If results show an infection in the abdomen, either drainage of the infection by the placement of tubes or surgery may be necessary. The physician will administer anti-autoimmune drugs or biologics as well to modify the body's aggressive immune response to microbes, which leads to sepsis.

Treatment for sepsis or severe sepsis/septic shock may further include early goal directed therapy, antibiotic, a vasopressor, such as norepinephrine and dopamine, a steroid, such as corticosteroids, insulin, painkillers, sedatives, oxygen, cerebrospinal fluid, and intravenous fluid to the subject. For application of these therapies, a central venous catheter and an arterial catheter may be used. Other hemodynamic variables (such as cardiac output, mixed venous oxygen saturation, or stroke volume variation) may also be used.

Treatment of organ dysfunction may include hemodialysis in kidney failure, mechanical ventilation in pulmonary dysfunction, transfusion of blood products, and drug and fluid therapy for circulatory failure. Ensuring adequate nutrition may further be required by enteral feeding, but if necessary by parenteral nutrition during a prolonged illness.

### H. IMMUNOASSAYS TO MEASURE MARKERS

The methods described above quantify levels of the following combination of markers selected from the group consisting of soluble CD25 (sCD25) and pancreatic stone protein (PSP) and sCD25 and procalcitonin (PCT).

The markers, i.e., sCD25, PSP, and PCT, may be analyzed for the methods described above using an immunoassay. The presence or amount of marker can be determined using antibodies that specifically bind to marker. Any immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA. Specific immunological binding of the antibody to the marker can be detected via direct labels, such as fluorescent or luminescent tags, metals and radionuclides attached to the antibody or via indirect labels, such as alkaline phosphatase or horseradish peroxidase.

The use of immobilized antibodies or fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test biological sample and then processed quickly through washes and detection steps to generate a measurable signal, such as a colored spot.

The Sandwich ELISA measures the amount of antigen between two layers of antibodies (i.e. capture and a detection antibody). The marker, i.e., sCD25, PSP, and PCT, to be measured may contain at least two antigenic sites capable of binding to antibody. Either monoclonal or polyclonal antibodies may be used as the capture and detection antibodies in the sandwich ELISA.

Generally, at least two antibodies are employed to separate and quantify the marker, i.e., sCD25, PSP, or PCT, in a test sample. More specifically, the at least two antibodies bind to certain epitopes of the marker forming an immune complex which is referred to as a “sandwich”. One or more antibodies can be used to capture the marker in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, both antibodies binding to their epitope may not be diminished by the binding of any other antibody in the assay to its respective epitope. In other words, antibodies may be selected so that the one or more first antibodies brought into contact with a test sample suspected of containing the marker do not bind to all or part of an epitope recognized by the second or subsequent antibodies, thereby interfering with the ability of the one or more second detection antibodies to bind to the marker.

In a preferred embodiment, a test sample suspected of containing the marker, i.e., sCD25, PSP, or PCT, can be contacted with at least one first capture antibody (or antibodies) and at least one second detection antibodies either simultaneously or sequentially. In the sandwich assay format, a test sample suspected of containing the marker is first brought into contact with the at least one first capture antibody that specifically binds to a particular epitope under conditions which allow the formation of a first antibody-marker complex. If more than one capture antibody is used, a first multiple capture antibody-marker complex is formed. In a sandwich assay, the antibodies, preferably, the at least one capture antibody, are used in molar excess amounts of the maximum amount of marker expected in the test sample.

Optionally, prior to contacting the test sample with the at least one first capture antibody, the at least one first capture antibody can be bound to a solid support which facilitates the separation the first antibody-marker complex from the test sample. Any solid support known in the art can be used, including but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind the marker. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample suspected of containing the marker is brought into contact with the at least one first capture antibody, the test sample is incubated in order to allow for the formation of a first capture antibody (or multiple antibody)-marker complex. The incubation can be carried out at a pH of from about 4.5 to about 10.0, at a temperature of from about 2° C. to about 45° C., and for a period from at least about one (1) minute to about eighteen (18) hours, from about 2-6 minutes, or from about 3-4 minutes.

After formation of the first/multiple capture antibody-marker complex, the complex is then contacted with at least one second detection antibody (under conditions which allow for the formation of a first/multiple antibody-marker second antibody complex). If the first antibody-marker complex is contacted with more than one detection antibody, then a first/multiple capture antibody-marker-multiple antibody detection complex is formed. As with first antibody, when the at least second (and subsequent) antibody is brought into contact with the first antibody-marker complex, a period of incubation under conditions similar to those described above is required for the formation of the first/multiple antibody-marker-second/multiple antibody complex. Preferably, at least one second antibody contains a detectable label. The detectable label can be bound to the at least one second antibody prior to, simultaneously with or after the formation of the first/multiple antibody-marker-second/multiple antibody complex. Any detectable label known in the art can be used.

### I. KITS FOR PERFORMING THE METHOD

Provided herein is a kit, which may be used for performing the methods described above. The kit may provide (1) at least one reagent capable of specifically binding the marker, i.e., sCD25, PSP, or PCT, to quantify the levels of the marker, i.e., sCD25, PSP, or PCT, in a biological sample isolated from a subject and (2) a reference standard indicating levels of the marker, wherein at least one reagent comprises at least one antibody capable of specifically binding the marker. The kit may further comprise at least one additional reagent capable of binding at least one additional biomarker selected from the group consisting of sCD25, PSP, and PCT to quantify the concentration of the at least one additional biomarker in the biological sample and a reference standard indicating the at least one additional biomarker in the biological sample, and a reference standard indicating a reference concentration of the at least one additional biomarker of sepsis or SIRS with infection or severe sepsis/septic shock, as discussed above.

The kit may comprise the antibody and a means for administering the antibody. The kit can further comprise instructions for using the kit and conducting the analysis, monitoring, or treatment.

The kit may also comprise one or more containers, such as vials or bottles, with each container containing a separate reagent. The kit may further comprise written instructions, which may describe how to perform or interpret an analysis, monitoring, treatment, or method described herein.

For example, the kit can comprise instructions for assaying the test sample for sCD25, PSP, or PCT by immunoassay, e.g., chemiluminescent microparticle immunoassay. The instructions can be in paper form or computer-readable form, such as a disk, CD, DVD, or the like. The antibody can be a sCD25, PSP, or PCT capture antibody and/or sCD25, PSP, or PCT detection antibody. Alternatively or additionally, the kit can comprise a calibrator or control, e.g., purified, and optionally lyophilized, (e.g., sCD25, PSP, or PCT), and/or at least one container (e.g., tube, microtiter plates or strips, which can be already coated with an anti-sCD25, PSP, or PCT monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve or a reference standard for purposes of quantifying sCD25, PSP, or PCT.

Any antibodies, which are provided in the kit, such as recombinant antibodies specific for sCD25, PSP, or PCT, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a blood sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution.

If desired, the kit can contain a solid phase, such as a magnetic particle, bead, test tube, microtiter plate, cuvette, membrane, scaffolding molecule, film, filter paper, a quartz crystal, disc or chip. The kit may also include a detectable label that can be or is conjugated to an antibody, such as an antibody functioning as a detection antibody. The detectable label can for example be a direct label, which may be an enzyme, oligonucleotide, nanoparticle, chemiluminophore, fluorophore, fluorescence quencher, chemiluminescence quencher, or biotin. Kits may optionally include any additional reagents needed for detecting the label.

If desired, the kit can further comprise one or more components, alone or in further combination with instructions, for assaying the test sample for another analyte, which can be a biomarker, such as a biomarker of cancer. Examples of analytes include, but are not limited to sCD25, PSP, or PCT, and fragments of sCD25, PSP, or PCT as well other analytes and biomarkers discussed herein, or otherwise known in the art. In some embodiments one or more components for assaying a test sample for sCD25, PSP, or PCT enable the determination of the presence, amount or concentration of sCD25, PSP, or PCT. A sample, such as a serum sample, can also be assayed for sCD25, PSP, or PCT using TOF-MS and an internal standard.

The kit (or components thereof), as well as the method of determining the concentration of sCD25, PSP, or PCT in a test sample by an immunoassay as described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., analyte antibody or capture antibody) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours), an automated or semi-automated format (e.g., ARCHITECT® and any successor platform, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT® and any successor platform).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STATED®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the detection antibody labeled with alkaline phosphatase (or other label). Within the fluid pouch of the cartridge is an aqueous reagent that includes p-aminophenol phosphate.

In operation, a sample suspected of containing sCD25, PSP and/or PCT is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between the first capture antibody, sCD25, PSP and/or PCT, and the labeled second detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of sCD25, PSP and/or PCT in the sample by means of an embedded algorithm and factory-determined calibration curve.

The present invention has multiple aspects, illustrated by the following non-limiting examples.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples which are merely intended only to illustrate some aspects and embodiments of the disclosure, and should not be viewed as limiting to the scope of the disclosure. The disclosures of all journal references, U.S. patents and publications referred to herein are hereby incorporated by reference in their entireties.

### EXAMPLES

### Example 1

### Materials and Methods

The following is a description of the materials and methods used in the below-identified Examples 2-4.

With respect to the study population, all admissions to the general intensive care unit (ICU) (17 beds) and high dependency unit (HDU) (8 beds) at Brighton and Sussex Hospitals NHS trust between August 2010 and January 2011 were sought to be enrolled. Patients were excluded if they were under 18 years of age or where it was not possible obtain patient consent or consultee approval to enrol the patient within 6 hours of admission. Baseline characteristics of the patients including demographic information, the Sequential Organ Failure Assessment (SOFA) score in the first 24 hours, comorbidities, site and type of infection and blood tests were collected. Patients were followed up until death or discharge from the ICU/HDU. A blood sample was collected from patients within 6 hours of their admission to the unit. Samples were put into sodium citrate tubes and centrifuged. The serum was stored at −80° C. until analysis.

Categorisation of subjects was made independently by at least two assessors who were blind to the biomarker results. Any disagreements were resolved by discussion. Interrater reliability between the assessors was high (Cohen's kappa 0.8).

With respect to biomarker measurements, levels of sCD25 were measured by ELISA using the Human IL-2 sRa (sCD25) OptEIA Set (Becton Dickenson, San Diego Calif.).

With respect to statistical analysis, continuous variables were described using the mean±SD for normally distributed data or the median [interquartile range (“IQR”)] for non-normally distributed data. Comparisons of group differences for continuous variables were made by one-way ANOVA or Mann-Whitney test as appropriate. Categorical data were described as the number of patients in each category with corresponding percentages. The significance of differences in proportions was tested by Chi-squared test. The performance of each marker in identifying sepsis or severe sepsis was assessed as area under a Receiver Operating Characteristic (ROC) curve. For each marker, ROC curves were used to derive cut-offs for sensitivity and specificity. To establish the potential for combinations of markers to improve identification of sepsis and severe sepsis, first univariate correlation (Spearman's rho) was used to establish the relationship between the markers. Since the majority of continuous variables were non-linear, associations between levels of each parameter and sepsis or severe sepsis were determined after dividing the values for each marker into quartiles. Stepwise logistic regression was then used to assess the impact of different combinations of markers on differentiation of sepsis from non-infectious SIRS. Statistical analyses were performed in SPSS 17.0 and Prism 5 (GraphPad Software Inc). All p values were two-sided and statistical significance was set at an α-value of 0.05.

### Example 2

### Base-Line Characteristics and Outcome of the Study Population

Between August 2010 and January 2011, 486 patients were admitted to the HDU/ICU. For 267 patients, no study blood sample could be obtained within 6 hours of admission because consultee approval could not be obtained in time. These patients did not enter the study. Of the 219 patients enrolled, 34 patients were admitted to the HDU and 185 patients were admitted to the ICU. The median age [IQR] of the patients was 65.9[52.0-76] years. 93 patients (42%) were female. 20 patients (9.1%) died on the ICU. 115 patients (52.5%) were surgical patients. The median length of stay was 3[1-6] days.

201 out of 219 patients (91.8%) met two or more of the SIRS criteria in the first 24 hours of admission to ICU. 87 out of 201 patients with SIRS (41.3%) were deemed to have an infective aetiology and thus had sepsis, while 75 patients (37.3%) were deemed not to have infection and were thus classified as having non-infectious SIRS. 36 patients could not be classified by clinical review as having sepsis or non-infectious SIRS (17.9%) and were excluded from further analysis. This group included 4 patients with pancreatitis. Demographic and clinical features of the different patient groups are shown in Table 1. Among patients with a systemic inflammatory response, those with organ failure were less likely to be on the HDU, less often to be post-surgical patients and had higher SOFA scores. Overall, patients with sepsis and non-infectious SIRS were similar in terms of age, gender and severity of illness.

TABLE 1
Base-line characteristics of the 179 patients who could be classified on basis of sepsis/SIRS criteria

| | | | NO-INFECTIVE SIRS | | | | SEPSIS | | | |
|---|---|---|---|---|---|---|---|---|---|---|
| | | | WITHOUT | | WITH | | WITHOUT | | WITH | |
| VARIABLE | NO SIRS | | ORGAN FAILURE | | ORGAN FAILURE | | ORGAN FAILURE | | ORGAN FAILURE | |
| N | 17 | | 25 | | 50 | | 11 | | 76 | |
| AGE (YEARS) | 72.1 | [61.5-75.4] | 63.3 | [51.2-71.5] | 62.6 | [51.0-74.5] | 65.1 | [42.6-73.4] | 66.7 | [54.3.-75.8] |
| FEMALE, N | 5 | (28%) | 11 | (44%) | 21 | (42%) | 6 | (54%) | 30 | (39%) |
| (%) | | | | | | | | | | |
| HDU, N (%) | 6 | (33%) | 11 | (44%) | 4 | (8%) | 4 | (36% | 4 | (5%) |
| SURGICAL | 14 | (78%) | 20 | (80%) | 19 | (38%) | 10 | (91%) | 36 | (47%) |
| SOFA SCORE | 4 | [3-7] | 1 | [1-2] | 6 | [3-8] | 2 | [1-3] | 7 | [5-10] |
| DAY 1 | | | | | | | | | | |

20 patients (9.1%) died during admission to HDU/ICU. Among patients with severe sepsis (i.e., sepsis patients with organ failure), 12 patients (16%) died compared with 4 patients (8%) with severe non-infectious SIRS (i.e., non-infectious SIRS patients with organ failure) (Table 2). Patients with sepsis on admission to ICU had a longer median length of stay than patients with non-infectious SIRS (4.05 vs 2.35 days (p=0.01)).

TABLE 2
Outcome of the patients

| | | NON-INFECTIOUS SIRS | | | | SEPSIS | | |
|---|---|---|---|---|---|---|---|---|
| | | WITHOUT | | WITH | | WITHOUT | WITH | |
| VARIABLE | NO SIRS | ORGAN FAILURE | | ORGAN FAILURE | | ORGAN FAILURE | ORGAN FAILURE | |
| N | 17 | 25 | | 50 | | 11 | 76 | |
| LENGTH OF | 2.8 [1.3-8.3] | 1.3 | [0.3-3.1] | 2.4 | [1.1-5.1] | 1.8 [1.3-10] | 4.1 | [2.4-7.8] |
| STAY ON ICU | | | | | | | | |
| MORTALITY | 0 | 1 | (4%) | 4 | (8%) | 0 | 12 | (16%) |
| ON ICU | | | | | | | | |

The characteristics of patients with sepsis are summarized in Table 3. Most of the sepsis patients had a focus of infection either in the respiratory tract or abdomen. Infection was microbiologically proven in 33 out of 87 patients with sepsis (38%) and diagnosed on radiological or clinical grounds for the remainder. Of the 72 patients with non-infectious SIRS, 29 were due to a post-surgical-related cause (i.e., maxofacial, urological), 11 were due to a cardiac-related cause (i.e., surgery or out-of-hospital cardiac arrest), and 9 were due to trauma or a medical-related cause (i.e., drug overdose, GI haemorrhage, diabetic ketoacidosis, acute asthma, seizures, pulmonary embolism, dehydration). Out of the 9 severe sepsis patients who had viral infection, 7 patients had proven influenza A infection and may also have had a bacterial superinfection that was not proven microbiologically, 1 patient had parainfluenza and 1 patient had “respiratory syncytial virus” (RSV) infection. One of the patients with severe sepsis had complicating polytrauma and thus it was not possible to determine a single anatomical focus.

TABLE 3
Microbiological and infection characteristics of the sepsis patients.

| | | | |
|---|---|---|---|
| | NON- | | |
| | SEVERE | SEVERE | |
| | SEPSIS | SEPSIS | TOTAL |
| VARIABLE | N = 11 | N = 76 | N = 87 |

ASSESSMENT OF INFECTION

| | | | |
|---|---|---|---|
| MICROBIOLOGICALLY | 4 (36%) | 29 (38%) | 33 (38%) |
| PROVEN | | | |
| GRAM-POSITIVE | 2 | 4 | 6 |
| BACTERIAL | | | |
| GRAM-NEGATIVE | 2 | 15 | 17 |
| BACTERIAL | | | |
| POLYMICROBIAL | 0 | 1 | 1 |
| VIRAL | 0 | 9^{A} | 9 |
| RADIOLOGICAL | 0 | 8 | 8 |
| DIAGNOSIS | | | |
| CLINICAL DIAGNOSIS | 7 | 39 | 46 |
| BACTERAEMIC | 2 | 5 | 7 |

FOCUS OF INFECTION

| | | | |
|---|---|---|---|
| RESPIRATORY TRACT | 1 | 32 | 33 |
| ABDOMEN | 8 | 30 | 38 |
| URINARY TRACT | 0 | 5 | 5 |
| SKIN AND SOFT TISSUE | 2 | 5 | 7 |
| OTHER | 0 | 3 | 3 |

### Example 3

### Biomarker Levels

C-reactive protein (CRP) was measured for clinical purposes in 143 of the 219 patients, however, the performance of CRP as a biomarker was not assessed. While levels of CRP were markedly higher in patients with sepsis than in patients with non-infectious SIRS (146 [105-203] ng/mL vs 9.5 [3.4-19.9] ng/mL, (p<0.001)), there was no difference in CRP depending on severity of illness.

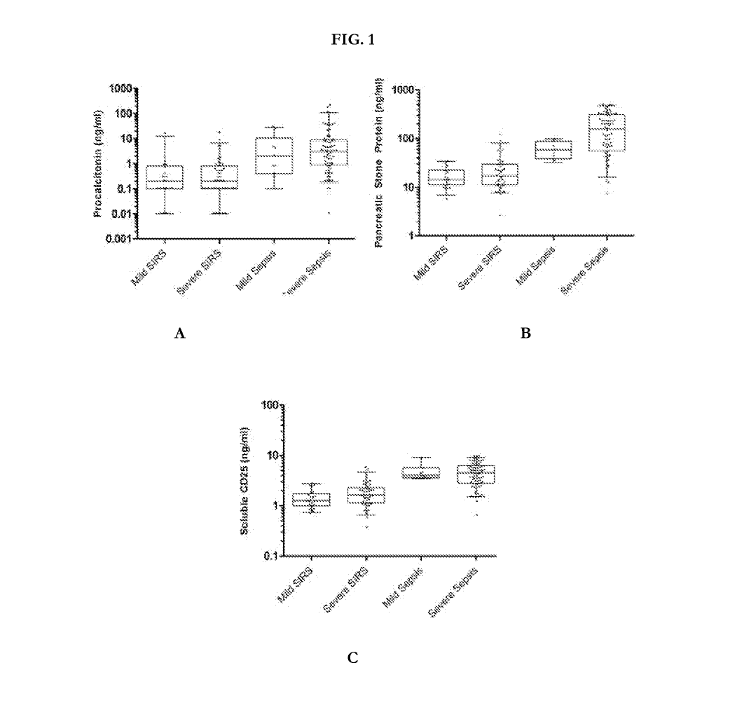

The levels of the various biomarkers in the blood samples taken from each of the 219 patients were measured (FIG. 1). Levels of procalcitonin (PCT) (3.1[0.8-3.9] ng/mL vs 0.2 [0.1-0.8] ng/mL, (p<0.001)), soluble CD25 (sCD25) (4.5 [3.0-6.1] ng/mL vs 1.5 [1.1-2.1] ng/mL, (p<0.001)), and Pancreatic Stone Protein (PSP) (116 [50-216] ng/mL vs 16.5 [11.1-27.9] ng/mL, (p<0.001)) were higher in patients with sepsis than in patients with non-infectious. PSP was the only marker for which levels were significantly higher in patients with severe sepsis than patients with mild sepsis (157 [56-310] ng/mL vs 59 [39-88] ng/mL, (p=0.01)).

These differences were reflected in the ROC analysis from which the AUCs for differentiating sepsis from non-infectious SIRS were as follows: PCT 0.840 (0.778-0.901), sCD25 0.902 (0.854-0.949), and PSP 0.927 (0.887-0.968). Although AUCs for PSP and sCD25 were higher than for PCT in differentiating sepsis from non-infectious SIRS, these differences were not statistically significant.

Arguably the most clinically important comparison in this group of patients was the differentiation of patients with severe sepsis or septic shock from patients with severe non-infectious SIRS. The performance of each marker was therefore assessed in 126 patients with SIRS and organ failure (76 severe sepsis or septic shock and 50 severe SIRS patients) (Table 4). In this comparison, again PSP, sCD25 and PCT all performed well with AUCs over 0.8.

TABLE 4
Diagnostic performance of different markers

| | CUT | | | | |
|---|---|---|---|---|---|
| MARKER | OFF | SENSITIVITY | SPECIFICITY | AUC | 95% CI |
| PCT | 1.0 | 70% | 85% | 0.84 | 0.77-0.91 |
| PSP | 30 | 88% | 78% | 0.91 | 0.86-0.96 |
| sCD25 | 2.5 | 87% | 65% | 0.87 | 0.81-0.93 |

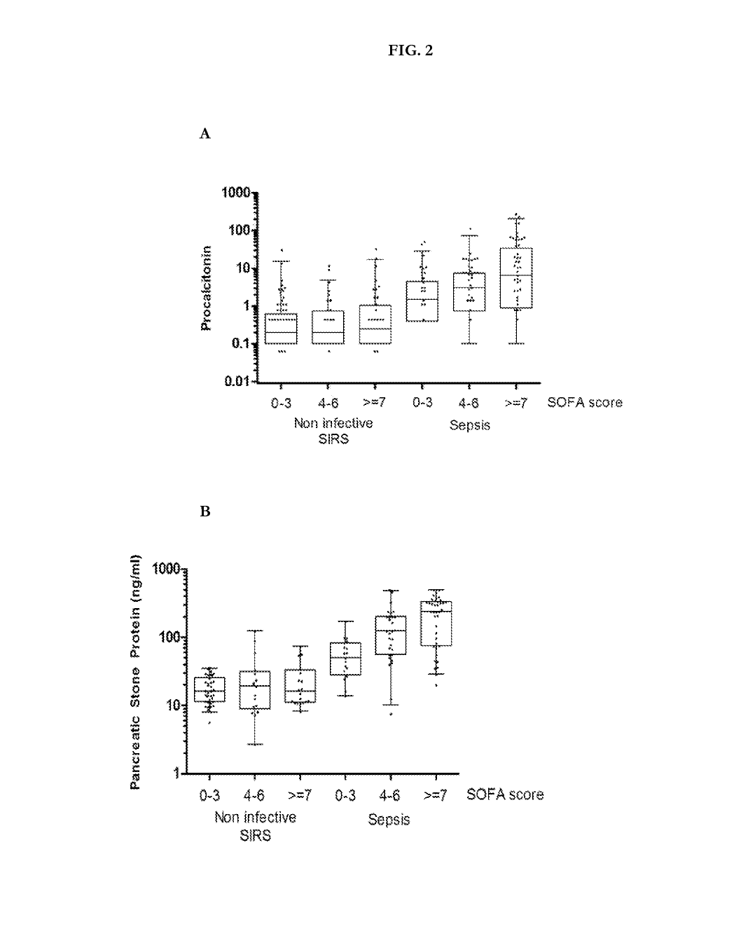

To look more closely at the ability of biomarkers to reflect severity of sepsis patients, non-infectious SIRS patients and sepsis patients were grouped according to SOFA score with severity defined as mild (SOFA score between 0-3) moderate (SOFA score between 4-6) and severe (SOFA greater than or equal to 7). For both PCT and PSP, there was a clear relationship between blood level and severity of sepsis, but not severity of non-infectious SIRS (FIG. 2). Focusing on patients with SOFA scores greater than or equal to 7, PSP may be superior in the discrimination of sepsis from non-infectious SIRS with an AUC of 0.955 (0.909-1) vs 0.837 (0.732-0.941). However, this difference was not statistically significant.

### Example 4

### Evaluation of Combined Predictive Value of Biomarkers

Calculation of correlation coefficients for the different variables revealed a marked degree of correlation between many of the markers. Correlation coefficients greater than 0.5 are shown in bold in Table 5. Correlation coefficients for PSP and PCT with sCD25 were between 0.55 and 0.6 (Table 5). In logistic regression analysis, addition of sCD25 improved the performance of both PCT and PSP by improving the AUC of these markers alone from 0.84 and 0.91 to 0.89 (0.83-0.95) and 0.94 (0.90-0.98), respectively. No improvement in the performance was observed with any other combination of markers

TABLE 5
Correlation between biomarkers.

| | | |
|---|---|---|
| PSP | sCD25 | PCT |

| | | | |
|---|---|---|---|
| sCD25 | RHO | 0.584 | |
| | P | 0.00 | |
| PCT | RHO | 0.591 | 0.546 |
| | P | 0.00 | 0.00 |

We claim:

1. A method for identifying and treating a subject with sepsis or severe sepsis/septic shock the method comprising:
(a) obtaining a biological sample comprising blood from the subject;
(b) determining the level of soluble CD25 (sCD25) and pancreatic stone protein (PSP) in the biological sample from the subject;
(c) comparing the levels of sCD25 and PSP in the biological sample to reference levels of sCD25 and PSP;
(d) identifying the subject as having sepsis or severe sepsis if the levels of sCD25 and PSP in the biological sample are greater than the reference levels of sCD25 and PSP; and
(e) administering a sepsis treatment regimen to the subject identified as having sepsis or severe sepsis.

2. The method of claim 1, wherein the reference levels of sCD25 and PSP are the levels of sCD25 and PSP in a control sample from non-infectious SIRS patients or septic shock patients.

3. The method of claim 1, wherein the reference levels of sCD25 and PSP are the sCD25 and PSP cutoff values determined by a receiver operating curve (ROC) analysis from biological samples of a patient group.

4. The method of claim 1, wherein the reference levels of sCD25 and PSP are the sCD25 and PSP cutoff values determined by a quartile analysis of biological samples of a patient group.

5. The method of claim 4, wherein the sCD25 and PSP cutoff values are about 2 ng/mL for sCD25 and about 25 ng/mL in serum for PSP.

6. The method of claim 1, further comprising determining the level of at least one additional biomarker of sepsis in the biological sample, and comparing the level of the at least one additional biomarker of sepsis to a reference concentration value for the at least one biomarker of sepsis.

7. The method of claim 6, wherein the additional biomarker of sepsis is procalcitonin (PCT).

8. The method of claim 1, further comprising determining the level of procalcitonin (PCT) in the biological sample and comparing the level of PCT in the biological sample to a reference level of PCT, wherein levels of sCD25, PSP and PCT in the biological sample greater than the reference levels of sCD25, PSP and PCT identifies the subject as having sepsis or severe sepsis.

9. The method of claim 1, wherein the subject is a human.

10. The method of claim 1, wherein the biological sample of a subject is selected from a tissue sample, bodily fluid, whole blood, plasma, serum, urine, bronchoalveolar lavage fluid, and a cell culture suspension or fraction thereof.

11. The method of claim 1, wherein the biological sample of a subject is blood plasma or blood serum.

12. The method of claim 1, wherein determining the levels of sCD25 and PSP comprises an immunological method with molecules binding to sCD25 and PSP.

13. The method of claim 12, wherein the molecules binding to sCD25 and PSP comprises at least one antibody capable of specifically binding sCD25 or PSP.

14. The method of claim 5, wherein levels sCD25 and PSP are above the cutoff values and indicates the individual is suffering from infectious systemic inflammatory response (SIR).

15. The method of claim 1, wherein the sepsis treatment regimen comprises administering at least one of an antibiotic, a vasopressor, a steroid, insulin, painkillers, sedatives, oxygen, cerebrospinal fluid, and intravenous fluid to the subject.

16. A method of providing a diagnosis of a subject having sepsis, the method comprising the steps of:
(a) obtaining a biological sample comprising blood from the subject;
(b) determining the level of soluble CD25 (sCD25) and pancreatic stone protein (PSP) in the biological sample from the subject;
(c) comparing the levels of sCD25 and PSP in the biological sample to reference levels of sCD25 and PSP; and
(d) providing a diagnosis of a subject having sepsis or severe sepsis/septic shock if the levels of sCD25 and PSP in the biological sample are greater than the reference levels of sCD25 and PSP.

17. A method of determining the severity of sepsis in a subject; the method comprising:
(a) obtaining a biological sample comprising blood from the subject;
(b) determining the level of soluble CD25 (sCD25) and pancreatic stone protein (PSP) in the biological sample from the subject;
(c) comparing the levels of sCD25 and PSP in the biological sample to reference levels of sCD25 and PSP; and
(d) correlating the levels of sCD25 and PSP in the biological sample with severity of sepsis in the subject wherein if the levels of sCD25 and PSP in the biological sample are higher than the reference level of sCD25 and PSP in the biological sample, the subject is determined to have increased severity of sepsis.

18. A method of monitoring the progression of sepsis in a subject, the method comprising:
(a) obtaining a biological sample comprising blood from the subject;
(b) determining the level of soluble CD25 (sCD25) and pancreatic stone protein (PSP) in the biological sample from the subject; and
(c) correlating the levels of sCD25 and PSP with progression of sepsis in the subject wherein if the levels of sCD25 and PSP are higher as compared to the levels of sCD25 and PSP in an earlier biological sample from the subject, the subject is identified as having progression of sepsis.

19. A method for the diagnosis, prognosis and/or risk stratification of sepsis or severe sepsis/septic shock in a subject having or suspected of sepsis or severe sepsis/septic shock, the method comprising the step of detecting increased levels of soluble CD25 (sCD25) and pancreatic stone protein (PSP) in the subject relative to a control subject not having sepsis or severe sepsis/septic shock.

20. The method of claims 16-19, further comprising administering a sepsis treatment regimen.

21. A kit for performing the method of claim 1, the kit comprising:
(a) at least one reagent capable of specifically binding sCD25 or PSP to quantify the levels of sCD25 or PSP in the biological sample of a subject; and
(b) a reference standard indicating reference levels of sCD25 and PSP.

22. The kit of claim 21, wherein the at least one reagent comprises at least one antibody capable of specifically binding sCD25 or PSP.

23. The kit of claim 21, further comprising at least one additional reagent capable of binding at least one additional biomarker of sepsis or severe sepsis/septic shock in the biological sample to quantify the concentration of the at least one additional biomarker in the biological sample, and a reference standard indicating a reference concentration of the at least one additional biomarker of sepsis or severe sepsis/septic shock in the biological sample.

24. A method for identifying and treating a subject with sepsis or severe sepsis/septic shock the method comprising:
(a) obtaining a biological sample comprising blood from the subject;
(b) determining the level of soluble CD25 (sCD25) and procalcitonin (PCT) in the biological sample from the subject;
(c) comparing the levels of sCD25 and PCT in the biological sample to reference levels of sCD25 and PCT;
(d) identifying the subject as having sepsis or severe sepsis if the levels of sCD25 and PCT in the biological sample are greater than the reference levels of sCD25 and PCT; and
(e) administering a sepsis treatment regimen to the subject identified as having sepsis or severe sepsis.

25. The method of claim 1, wherein the reference levels of sCD25 and PCT are the levels of sCD25 and PCT in a control sample from non-infectious SIRS patients or septic shock patients.

26. The method of claim 1, wherein the reference levels of sCD25 and PCT are the sCD25 and PCT cutoff values determined by a receiver operating curve (ROC) analysis from biological samples of a patient group.

27. The method of claim 1, wherein the reference levels of sCD25 and PCT are the sCD25 and PCT cutoff values determined by a quartile analysis of biological samples of a patient group.

28. The method of claim 4, wherein the sCD25 and PCT cutoff values are about 2 ng/mL for sCD25 and about 1 ng/mL in serum for PCT.

29. The method of claim 1, further comprising determining the level of at least one additional biomarker of sepsis in the biological sample, and comparing the level of the at least one additional biomarker of sepsis to a reference concentration value for the at least one biomarker of sepsis.

30. The method of claim 6, wherein the additional biomarker of sepsis is pancreatic stone protein (PSP).

31. The method of claim 1, further comprising determining the level of pancreatic stone protein (PSP) in the biological sample and comparing the level of PSP in the biological sample to a reference level of PSP, wherein levels of sCD25, PCT and PSP in the biological sample greater than the reference levels of sCD25, PCT and PSP identifies the subject as having sepsis or severe sepsis.

32. The method of claim 1, wherein the subject is a human.

33. The method of claim 1, wherein the biological sample of a subject is selected from a tissue sample, bodily fluid, whole blood, plasma, serum, urine, bronchoalveolar lavage fluid, and a cell culture suspension or fraction thereof.

34. The method of claim 1, wherein the biological sample of a subject is blood plasma or blood serum.

35. The method of claim 1, wherein determining the levels of sCD25 and PCT comprises an immunological method with molecules binding to sCD25 and PCT.

36. The method of claim 12, wherein the molecules binding to sCD25 and PCT comprises at least one antibody capable of specifically binding sCD25 or PCT.

37. The method of claim 5, wherein levels sCD25 and PCT are above the cutoff values and indicates the individual is suffering from infectious systemic inflammatory response (SIR).

38. The method of claim 1, wherein the sepsis treatment regimen comprises administering at least one of an antibiotic, a vasopressor, a steroid, insulin, painkillers, sedatives, oxygen, cerebrospinal fluid, and intravenous fluid to the subject.

39. A method of providing a diagnosis of a subject having sepsis, the method comprising the steps of:
(a) obtaining a biological sample comprising blood from the subject;
(b) determining the level of soluble CD25 (sCD25) and procalcitonin (PCT) in the biological sample from the subject;
(c) comparing the levels of sCD25 and PCT in the biological sample to reference levels of sCD25 and PCT; and
(d) providing a diagnosis of a subject having sepsis or severe sepsis/septic shock if the levels of sCD25 and PCT in the biological sample are greater than the reference levels of sCD25 and PCT.

40. A method of determining the severity of sepsis in a subject; the method comprising:
(a) obtaining a biological sample comprising blood from the subject;
(b) determining the level of soluble CD25 (sCD25) and procalcitonin (PCT) in the biological sample from the subject;
(c) comparing the levels of sCD25 and PCT in the biological sample to reference levels of sCD25 and PCT; and
(d) correlating the levels of sCD25 and PCT in the biological sample with severity of sepsis in the subject wherein if the levels of sCD25 and PCT in the biological sample are higher than the reference level of sCD25 and PCT in the biological sample, the subject is determined to have increased severity of sepsis.

41. A method of monitoring the progression of sepsis in a subject, the method comprising:
(a) obtaining a biological sample comprising blood from the subject;
(b) determining the level of soluble CD25 (sCD25) and procalcitonin (PCT) in the biological sample from the subject; and
(c) correlating the levels of sCD25 and PCT with progression of sepsis in the subject wherein if the levels of sCD25 and PCT are higher as compared to the levels of sCD25 and PCT in an earlier biological sample from the subject, the subject is identified as having progression of sepsis.

42. A method for the diagnosis, prognosis and/or risk stratification of sepsis or severe sepsis/septic shock in a subject having or suspected of sepsis or severe sepsis/septic shock, the method comprising the step of detecting increased levels of soluble CD25 (sCD25) and procalcitonin (PCT) in the subject relative to a control subject not having sepsis or severe sepsis/septic shock.

43. The method of claims 16-19, further comprising administering a sepsis treatment regimen.

44. A kit for performing the method of claim 1, the kit comprising:
(a) at least one reagent capable of specifically binding sCD25 or PCT to quantify the levels of sCD25 or PCT in the biological sample of a subject; and
(b) a reference standard indicating reference levels of sCD25 and PCT.

45. The kit of claim 21, wherein the at least one reagent comprises at least one antibody capable of specifically binding sCD25 or PCT.

46. The kit of claim 21, further comprising at least one additional reagent capable of binding at least one additional biomarker of sepsis or severe sepsis/septic shock in the biological sample to quantify the concentration of the at least one additional biomarker in the biological sample, and a reference standard indicating a reference concentration of the at least one additional biomarker of sepsis or severe sepsis/septic shock in the biological sample.
